(19) United States
(12) Patent Application Publication
(10) Pub. No.: US 2011/0319284 A1
(43) Pub. Date: Dec. 29, 2011

(54) METHOD FOR DETERMINING THE PREDISPOSITION FOR CROHN'S DISEASE

(76) Inventors: Jan Wehkamp, Stuttgart (DE); Eduard Stange, Stuttgart (DE); Maureen Koslowski, Stuttgart (DE)

(21) Appl. No.: 13/141,315

(22) PCT Filed: Dec. 21, 2009
(86) PCT No.: PCT/EP2009/009176

§ 371 Date: Sep. 9, 2011

(30) Foreign Application Priority Data
Dec. 22, 2008 (DE) 10 2008 064 509.5

(51) Int. Cl. C12Q 1/68; C40B 30/04; C07H 21/04

(52) U.S. Cl. 506/9; 435/6.11; 536/24.31; 536/24.33

(57) ABSTRACT

A method is described for determining a predisposition of an organism for Crohn's Disease, especially Crohn's Disease of the small intestine. In this context, in a biological specimen of an organism, the presence or the absence of SNP's in at least one gene is determined, which codes for a protein associated with the Writ signaling pathway in Paneth cells. The gene, in this instance, may be selected from TCF4, LRP5, LRP6, GSK3A, GSK3B and TCF7. The present methods and systems also relate to primers and allele-specific probes to prove the presence or the absence of an SNP, diagnostic kits which have at least one such primer or one such allele-specific probe, as well as the use of certain SNP's for determining a predisposition of an organism for Crohn's Disease. The present method and systems also relate to a method for the differential diagnosis of inflammatory bowel diseases, for distinguishing Crohn's Disease and the other respective inflammatory or infectious intestinal diseases.

### RELATED APPLICATION INFORMATION

This application is a national-phase application based on international application PCT/EP2009/009176, filed on Dec. 21, 2009, which claims the benefit of and priority of German Patent Application No. 10 2008 064 509.5, which was filed in Germany on Dec. 22, 2008, the entire contents of all of which are expressly incorporated herein by reference.

### FIELD OF THE INVENTION

The present invention relates to a method for determining a predisposition of an organism for Crohn's Disease, in which the presence of single nucleotide polymorphisms (SNP) is determined from a biological specimen of the organism. Furthermore, the present invention relates to primers and allele-specific probes to prove the presence or absence of an SNP, diagnostic kits which have at least one such primer or one such allele-specific probe, as well as the use of certain SNP's for determining a predisposition of an organism for Crohn's Disease. Chronic inflammatory intestinal diseases include two different clinical pictures, colitis ulcerosa and Crohn's Disease. Both diseases lead to a persistent impairment of the quality of life, by severe inflammation reactions of the intestine, which are accompanied by severe diarrhea, rectal incontinence and additional complaints. Moreover, both diseases may be connected to an increase in the risk for intestinal cancer.

### BACKGROUND INFORMATION

In Western industrial states, both diseases appear at an high frequency of 150 sick people per 100,000 persons (Crohn's Disease) and 80 sick people per 100,000 persons (colitis ulcerosa). Furthermore, in the industrial as well as the developing countries, the tendency for the appearance of chronic inflammatory intestinal diseases has been increasing for decades.

The chronic inflammatory intestinal diseases mentioned are considered to be caused in a multifactorial manner, in this instance, genetic predisposition, various environmental factors as well as modified immune reactions being of importance, which lead to the destruction of the mucosal barrier and are triggered or sustained by the intestinal flora.

Thus, for example, mutations in the MDR1 gene, which codes for p-glycoprotein 170, have been connected to an increased risk of falling ill with ulcerative colitis. Also, a series of genetic factors is known which favor the occurrence of Crohn's Disease, such as mutations in the genes of NOD2, and OCTN1 and 2; (see Suchy et al.: “Inflammatory response gene polymorphisms and their relationship with colorectal cancer risk”, BMC Cancer 2008; 8:112; Okazaki et al.: “Contributions of IBDS, IL23R, ATGI6L1, and NOD2 to Crohn's disease risk in a population-based case-control study: Evidence of gene-gene interactions”, Inflamm. Bowel Dis. 2008; 14:1528-1541; Henckaerts L. et al.: “The role of genetics in inflammatory bowel disease”, Curr. Drug Targets 2008; 9(5):361-8).

All-in-all, one may see from this that there is a great need for a diagnostic arrangement, so as, on the one hand, to evaluate the risk of a patient of falling ill with a chronic inflammatory intestinal disease, using genetic diagnosis, so that behavioral adaptations may be recommended to the patient for reducing the risk of falling ill. On the other hand, it should also be possible, using genetic diagnosis, to diagnose the molecular causes of an individual chronic inflammatory intestinal disease, and to initiate a therapy adapted to it.

Several methods for the genetic diagnosis of inflammatory intestinal diseases are known from the related art.

Thus, WO 2004/083232 A2 discusses a method for the diagnosis of the genetic predisposition of an individual for a chronic inflammatory intestinal disease, in which SNP's are able to be identified, in the CCRL2 gene. Such mutations demonstrate a correlation with the appearance of colitis ulcerosa.

US 2006/0141478 A1 discusses a diagnostic measure in which the promoter variants of the NF-κB gene are diagnosed and correlated with the appearance of chronic inflammatory intestinal diseases, both Crohn's Disease and colitis ulcerosa.

However, the diagnostic methods, from the related art, without exception have limited meaningfulness. Thus, in the preliminary stages, the risk of falling ill with a chronic inflammatory intestinal disease, can be detected only inadequately. Also, for instance, by the diagnosis of promoter variants of the NF-κB gene, it is not possible to differentiate between different variants of chronic inflammatory intestinal diseases.

### SUMMARY OF THE INVENTION

With this as background, the exemplary embodiments and/or exemplary methods of the present invention are based on an object of improving existing diagnostic methods, by improving the predictive accuracy and simplifying the differentiation between various chronic inflammatory intestinal diseases.

This object is attained, according to the exemplary embodiments and/or exemplary methods of the present invention, by a method for determining the predisposition of an organism for Crohn's Disease in which, in a biological probe of the organism, the presence of SNP's is determined, which is characterized in that at least one SNP is determined in at least one gene which codes for a protein associated with the Wnt signaling pathway in Paneth cells (cells of Paneth).

If at least one SNP is present, the predisposition of the organism for the development of Crohn's Disease is certain.

Thus, the object is completely attained by the present invention.

By “SNP's” (single nucleotide polymorphisms), variations of individual base pairs in a DNA strand are designated in comparison to the wild type in a certain population. SNP's represent ca. 90% of all genetic variants in the human genome, and occur no uniformly represented at certain regions in the genome. They represent mutations, i.e. genetic changes, which have succeeded to a certain extent in the gene pool of a population. In this context, the Snip's are able to appear as substitutions in which a base, such as cytosine is exchanged for another base, such as thymine, or as deletions or insertions.

In this context, Snip's always have one of two states, or very rarely one of several states, and are allelic ally inherited. The majority of the known Snip's relates to nonbonding regions in the genome, i.e. regions which lie either between genes or between exon regions of individual genes. Basically, these gene variants, in noncoding regions, may also affect regulatory sequences, such as promoters, enhancers or splicing sites, and thus have effects on the expression of genes. SNP's which affect the coding sequence directly may be quiescent, that is, the base exchange does not change the translation of the respective triplet code into the analogous amino acid, and thus has no effect on the peptide sequence. However, because of a different frequency of equivalent t-RNA's for specific base triplets, differences in the efficiency of the translation are able to come about, and quiescent SNP's may lead to a changed secondary structure of the mRNA (e.g. “hairpin”), and thus the expression of certain genes may be affected post-transcriptionally by quiescent SNP's. Some SNP's have coding function, that is, the different alleles lead to a different incorporation of an amino acid into the peptide being created (“missense SNP”), with the result that this function is able to be changed. If an SNP does not change the amino acid sequence of a protein, the SNP is usually “quiescent”.

The term “allele” in this case, and also in the specialty field, relates to alternative forms of genes or sections thereof. On homologous chromosomes, alleles occupy the same locus; this means that, if a subject has two identical alleles of a gene, this subject is homozygous for this allele, and if the subject has different alleles of a specific gene, this subject is heterozygous for the allele. In this context, alleles of a specific gene may differ from each other in a single nucleotide; moreover, an allele of a gene may also be present in a gene having one or more mutations or sequence variants. In the method according to the present invention, in each case one allele or both alleles may be investigated for the presence of the SNP.

In this instance, by a nucleotide of a nucleic acid, which may be present as DNA or RNA, one should understand adenine, cytosine, guanine, thymine, and in the case of RNA, uracil instead of thymine.

Within the scope of the exemplary embodiments and/or exemplary methods of the present invention, by “SNP” one should understand, in particular, a point mutation in the genome of an organism, which brings about an exchange of a base present at a certain position for a certain other base, according to the genetic norm prevailing in the respective species. In this context, usually, if in an SNP the same position is affected, the same “rs number” is always allocated; the polymorphism is then given as C/A/ or G corresponding to the example.

The SNP's, if they are present biallelically, are able to occur in three possible genotypes in the genome, namely in one of two possible homozygous forms (allele 1/allele 1 or allele 2/allele 2) or in an heterozygous form (allele 1/allele 2). Adjacent SNP's may be linked to each other to a different degree, i.e., to a certain percentage, they occur in the population in a certain combination only in common, and thereby form a so-called haplotype.

By “organism” one should understand, in this instance, especially mammals, particularly human beings.

By “biological specimen”, every specimen is understood, in this instance, which contains genetic material and which was taken from a patient, especially an human being, and which is then used in vitro for the purposes of the diagnostic method according to the present invention. In this context, the biological specimen includes both specimens of body fluids which contain cells, as for instance blood, saliva, etc., or from tissues, including organs.

“Linkage” and “coupling” at present describe the tendency of the genes, alleles, loci or genetic markers to be inherited in common as a result of their localization on the same chromosome. The expression “linkage disequilibrium” or “coupling disequilibrium” (also designated by “LD” below) refers to a more than random association between specific alleles at two marker loci within a certain population.

Generally, the coupling disequilibrium decreases with an increase in the physical distance of the loci.

The term “determine”, as is here used for the determination of the SNP's, refers to various ways and methods for the analysis of one or more SNP's at a certain site in the genome, the term including both the direct determination, i.e., for instance, the sequencing and the indirect determination, that is, for instance, the amplification and/or the hybridization.

In the present case, Crohn's Disease is also abbreviated to CD, originating from the English (Crohn's Disease), and chronic inflammatory intestinal disease to IBD, also an abbreviation originating with the English (inflammatory bowel disease).

Now, the inventors have recognized that it is surprisingly possible, with the aid of certain SNP's in the genomic regions, to diagnose a genetic predisposition for Crohn's Disease, specifically Crohn's Disease of the small intestine, from factors, of the Wnt signaling pathway in Paneth cells.

The intestinal barrier function is a central factor in the pathogenesis of chronic inflammatory bowel diseases, such as Crohn's Disease. In the human intestine, the epithelial cells, for one thing, form a physical barrier, for another thing, as a part of the innate immune defense, they produce an effective chemical as well as biological protective layer to prevent an invasion of microbes. In the small intestine, compared to the densely colonized large intestine, there are relatively few bacteria, but still, their number in the lumen reaches 10⁵-10⁷ per ml of fluid. In spite of this constant presence of microorganisms, there are only seldom infections or inflammatory reactions in healthy people. The antimicrobial products of Paneth cells play an important part in this connection. These cells occur specifically in the base of the crypts of the small intestine, which distinguishes them from the other well-differentiated cell types of the intestinal epithelium. They originate from multipotent adult stem cells, that lie between and/or over the Paneth cells, and that have the capability of self-regeneration, by definition. Daughter cells originating from them form a zone of precursor cells (transient amplifying cells) which, after 4-6 cell divisions, differentiate into the various post-mitotic cell types, the absorptive and enterendrocrine cells and the Becher cells and Paneth cells of the epithelium.

The Wnt signaling pathway occupies an important function in the obtaining of the intestinal stem cells as well as the differentiation of Paneth cells. In adult Paneth cells, its activity is indispensable in addition for the correct functioning of the cell, since it directly arranges for the expression of antimicrobial substances, of Paneth cell α-defensin HD-5 (DEFA5) and HD-6 (DEFA6).

In patients having Crohn's Disease of the small intestine, the expression of these substances is reduced, and is thus able to lead to a restriction in the intestinal immune defense in this region. A weakening of the protective barrier may enable bacteria to penetrate into the epithelium, and thus lead to the creation of an adaptive immune reaction as well as an inflammation. The specific reduction of HD-5 and HD-6 in Crohn's Disease of the small intestine may lead one to conclude that there is an important role of the Paneth cell in the creation of this disease, which is also substantiated by several genetic studies.

It is known, to be sure, for example, that Cohn's Disease patients have an impaired exocytose of the granola of Panetta cells with a certain polymorphism T300A of ATG16 autophagy related 16-like 1 (ATG16L1) gene (Cadwell et al., “A key role for autophagy and the autophagy gene Atgl611 in mouse and human intestinal Paneth cells”, Nature 2008; 456(7219):259.63). The granula of Paneth cells represent a type of pantry for antimicrobial substances and their unimpairment is essential for the secretion of these active substances.

One additional gene that is strongly associated with Crohn's Disease of the small intestine is the bacterial diagnostic molecule “Nucleotide-binding oligomerization domain containing 2” (NOD2), also known as CARD15. This muramyldipeptide receptor is present intracellularly in Paneth cells, and a frameshift mutation, which leads to a shortened form of the protein, occurs in connection with a particularly low expression of Paneth cell α-defensins (Wehkamp et al., “NOD2 (CARD15) mutations in Crohn's disease are associated with diminished mucosal α-defensin expression”, Gut 2004; 53:1658-64; Wehkamp et al.: “Reduced Paneth (alpha)-defensins in ileal Crohn's Disease”, PNAS 2005; 102:18129-34).

Wnt proteins are a family of secreted morphogens, which play an important part in the regulation of cell differentiation in the adult organism, as well as the differentiation during embryogenesis. The Wnt signaling pathway is induced by the binding of a protein of the Wnt family to frizzled among other cell surface receptors. This induction is favored by so-called LRP proteins (“low density lipoprotein receptor-related protein”), which also act as receptors. Because of the binding of the Wnt proteins to surface receptors, intracellularly, various signal transduction paths are activated, for example, via the protein disheveled. Thus, disheveled inhibits or inactivates a protein complex containing glycogen synthase kinase 3 (GSK3), which normally exposes β catenin to catabolism by the proteasome, and thus prevents its accumulation. Thereby, a cytoplasmic pool of the protein β-catenin is stabilized, is able to get into the nucleus, and there, in collaboration with transcription factors of the Lef/TCF family, activates a series of target genes.

To be sure, it was known that the differentiation of the Paneth grain cells, as well as the secretion of human α-defensin (HD) 5 and 6 by proteins of the family of TCF transcription factors is at least codetermined, and that in some people having fallen ill with Crohn's Disease a reduced expression of defensins, on the one hand, and Tcf4, on the other hand, may be determined, but it was not to be expected that certain genetic variations, associated with the genes of Wnt signaling pathways, would demonstrate a clear association with the occurrence of Crohn's Disease, especially Crohn's Disease of the small intestine.

The inventors of the present Application now have realized for the first time that genes, which take up important functions in the Wnt signaling pathway, demonstrate changes in connection with the disease. In their own investigations, the inventors were able to show that mutations in important genes of factors/proteins participating in the Wnt signaling pathways have an effect on it and, with that, have an effect on the development and the functioning of the Paneth cells.

In this connection, the new method has the advantage that, which may be in combination with the diagnosis of the presence or absence of known disease-associated SNP's, for instance, in the genes of NOD2, ATG16L1, DLG5, the predictive accuracy for the occurrence or non-occurrence of Crohn's Disease is increased.

The diagnostic method, now provided by the inventors, having the determination of SNP's in the Wnt signaling pathway also makes possible, therefore, a simplified differential diagnosis, since using the method according to the present invention, Crohn's Disease is then able to be differentiated more efficiently from bacterial infections, such as tuberculosis or yersiniosis. This advantageously, on the one hand, enables a rapid, positive diagnosis and, on the other hand, also a rapid, specific therapeutic approach.

Therefore, the exemplary embodiments and/or exemplary methods of the present invention also relate to a method for the differential diagnosis of inflammatory bowel diseases, for distinguishing Crohn's Disease and the other respective inflammatory and infectious intestinal diseases. In the method according: to the present invention, in a biological specimen of the organism, the presence of at least one single nucleotide polymorphisms (SNP) is also determined, that codes for a protein associated with the Wnt signaling pathway in Paneth cells.

Using the new method, it is possible, for the first time, to prognosticate the individually probable intensity of the disease. Thus, the new method may be used to prognosticate differentially the probability of the appearance of Crohn's Disease, with or without the involvement of the small intestine.

Furthermore, the method may explain the individual cause or possible later cause of Crohn's Disease. In this connection, a genetic defect involved in the path of creation of Crohn's Disease may be specifically identified, so that it may be determined exactly at which site of the participating Wnt signaling pathway a faulty regulation has occurred or will still occur.

Based on such a diagnosis, it is possible, for the first time, to treat Crohn's Disease causatively.

By contrast to this, using the data gathered by the new method, it is possible to conceive of and to carry out a therapy, for instance, a gene therapy, that is adjusted to the individual patient. In this instance, because of vectors, such as viruses inserted in the intestine, specifically the cells or tissues could be infected and transformed that are affected by Crohn's Disease. Consequently, a functioning signaling pathway could be produced and set to act against the development of Crohn's Disease.

In a particular variation of the method according to the present invention, at least one SNP is determined in at least one of the following genes: TCF4 (designated also as TCF7L2, HGNC (HUGO Gene Nomenclature Committee) ID: 11641), LRP5 (HGNC-ID: 6697), LRP6 (HGNC:6698), GSK3A (HGNC-ID:4616), GSK3B (HGNC-ID:4617), and TCF7 (designated also as TCF1, HGNC-ID:11639). (For the identification of the genes, see www.genenames.org).

By the “genes” named, at present always the coding sequence of the given gene as well as the intron sequences and the 5′ and 3′ untranslated/regulatory regions of the gene named are understood.

The inventors of the present Application present data with respect to Tcf4, the Wnt transcription factor, which directly induces HD5 and HD6, as well as with respect to low density lipoprotein receptor-related proteins LPR5 and LPR6, important receptor molecules of the signaling pathways, with the aid of which one could clearly show a connection between a change in genes, which code for proteins associated with the Wnt signaling pathway, and Crohn's Disease, especially of the small intestine. The data provided show that factors that codetermine the functioning of Paneth cells represent important candidates in the pathogenesis of Crohn's Disease.

Furthermore, the exemplary embodiments and/or exemplary methods of the present invention relates to primers and allele-specific probes and oligonucleotides to show the presence or the absence of an SNP, diagnostic kits which have at least one such primer or one such allele-specific probe, as well as the use of SNP's in at least one gene of the group TCF4, LRP5, LRP6, GSK3A, GSK3B and TCF7 for determining a predisposition of an organism for Crohn's Disease. In general, a polymorphism is able to be proved in the genomic DNA, in the mRNA or in the cDNA.

In this context, “primer” (or “probe” or “oligonucleotide probe”) means an oligonucleotide or a single-stranded sequence of nucleic acid which, in combination with DNA-replicating enzymes, for instance, DNA polymerase, are used as starting point, in order, for instance, with the aid of a so-called “template” DNA to amplify a certain region (particularly in the polymerase chain reaction (PCR); thus, with the aid of the primers, the specific DNA section, that is to be amplified, may be established. Probes are mostly furnished with a molecular (e.g. radioactive) marking, and are used in molecular-biological hybridization methods for the sequence-specific detection of DNA and RNA molecules. A sequence-specific primer/probe does: not hybridize with alleles of a gene locus which does not contain the sequence polymorphism for which the primer/the probe is specific.

The primers, which according to the exemplary embodiments and/or exemplary methods of the present invention are suitable for the sequence-specific detection of the SNP's, therefore include a sequence which may overlap the polymorphism or flanks it.

Thus, in a particular variation of the method according to the present invention, at least one SNP is detected, which is selected from the group including the SNP's having reference numbers rs3814570, rs10885394, rs10885395, rs682429, rs4988331, rs554734, rs312778, rs7302808, rs7136380, rs7308022, rs2417086, rs6488506, rs7294695, rs12320259, rs1231320, rs2284396, rs2302685, 12314349, rs12313200, rs11054701, rs11609634, rs2417085, rs10845494, rs1181334, rs10772542, rs10743980, rs7304561.

The SNP's listed are allocated to the following genes:

TABLE 1
Allocation of the SNP's to their genes by their rs numbers:

| TCF4 | LRP5 | LRP6 |
|---|---|---|
| rs3814570 | rs682429 | rs7302808 |
| rs10885394 | rs4988331 | rs7136380 |
| rs10885395 | rs554734 | rs7308022 |
| | rs312778 | rs2417086 |
| | rs2242339 | rs6488506 |
| | | rs7294695 |
| | | rs12320259 |
| | | rs12313200 |
| | | rs2284396 |
| | | rs2302685 |
| | | rs12314349 |
| | | rs11054701 |
| | | rs11609634 |
| | | rs2417085 |
| | | rs10845494 |
| | | rs1181334 |
| | | rs10772542 |
| | | rs10743980 |
| | | rs7304561 |

In the present case, the “rs” number means the reference number for the respective SNP; among the respectively given numbers, for instance, under http://www.ncbi.nlm.nih.gov/ under “Entrez SNP”, the corresponding sequences of the SNP's may be found, as well as additional information on them, such as, for instance, the chromosomes localization of the genes and their SNP's.

Thus, the SNP having the number rs3814570 relates to a polymorphism having a polymorphous exchange or substitution of the base cytosine (C) for another base, especially thymine (T), at position −2006 relative to transcription start codon in the promoter of the Tcf4 gene.

The SNP having the number rs10885394 refers to a substitution of the adenine by another base, particularly thymine, at position −1728 relative to the transcription start codon in the promoter of the Tcf4 gene, the SNP having the number rs10885395 refers to a substitution of the cytosine for another base, particularly thymine, at position −1248 relative to the transcription start codon in the promoter of the Tcf4 gene.

An overview of the positions of the SNP's as well as the exchanged bases in the genes described may be found in the following Table 2:

TABLE 2
Position of the SNP's in the genes Tcf4, LRP5 and LRP6

| | Base exchange | Position* | |
|---|---|---|---|
| TCF4 | | | |
| rs3814570 | C/T** | −2006 | 5′ assumed promoter |
| rs10885394 | A/T | −1728 | |
| rs10885395 | C/T** | −1248 | |
| LRP5 | | | |
| rs682429 | A/G | −864 | 5′ assumed promoter |
| rs4988331 | C/T | 5150 | |
| rs554734 | G/T | 112335 | |
| rs312778 | C/T | 28149 | |
| Rs2242339 | C/T | 98942 | |
| LRP6 | | | LRP6: opposite direction on |
| | | | strand |
| rs7302808 | C/T | −563 | 5′ assumed promoter |
| rs7136380 | C/T | −566 | |
| rs7308022 | A/G** | 13797 | |
| rs2417086 | A/G | 69556 | |
| rs6488506 | A/G | 101286 | |
| rs7294695 | C/G | 96051 | |
| rs12320259 | C/T | 119500 | |
| rs12313200 | A/G | 135079 | |
| rs2284396 | C/T | 144734 | |
| rs2302685 | C/T | 117771 | “missense”-mutation: V1062I |
| rs12313200 | G/A | 134754 | Tag SNP |
| rs12314349 | A/T | 131848 | Tag SNP |
| rs11054701 | C/T | 130513 | Tag SNP |
| rs11609634 | C/T | 109982 | Tag SNP |
| rs2417085 | C/T | 88294 | Tag SNP |
| rs10772542 | C/T | 25774 | Tag SNP |
| rs1181334 | G/T** | 52081 | Tag SNP |
| rs10845494 | C/T | 70770 | Tag SNP |
| rs10743980 | C/T | 6874 | Tag SNP |
| rs7304561 | A/G | −2736 | |

*position relative to transcription start
**“original” allele not known

The original alleles are marked in bold, but are not always the ones occurring most frequently

In one specific embodiment of the method according to the present invention, for determining the predisposition of a patient/subject for the development of Crohn's Disease, a biological specimen of the patient/subject is tested for the SNP's cited herein, or rather the presence of the various SNP alleles is detected, and the genetic information thus acquired is compared to appropriate control data and/or data banks, which contain data with respect to polymorphisms in the genes cited herein; and based on this comparison, the probability of the development of Crohn's Disease is calculated.

Within the scope of the new method, the detection of the SNP's may be carried out using various methods known to one skilled in the art. In particular, it may be preferred in this instance if the detection of the SNP's, in the present method, takes place using the MassARRAY® technique, sequencing or an hybridization technique, such as a DANN, RNA or protein chip. These methods each have individual advantages.

Thus, the “MassARRAY®” method makes it possible to detect specifically comparatively large quantities of SNP's in a cost-effective manner. Sequencing, by contrast, offers the possibility of classifying the SNP's in the individual genetic background of the respective 3′ and 5′ flanking sequences. The hybridization techniques offer the possibility of investigating a plurality of SNP's simultaneously. Furthermore, these techniques are extensively established.

The detection methods to be used, in the method according to the present invention, may be used, in this context, to detect polymorphisms in a biological specimen in cells that are intact (such as in situ hybridization), or in extracted DNA (for instance, by Southern Blot Hybridizations). In allele-specific hybridization, in this context, probes or primers may be used which overlap the polymorphism site and have ca. 5, 10, 20, 25 or 30 nucleotides around the SNP.

In one exemplary method, a plurality of probes, which are able to bind specifically to the allele variants, are immobilized on a fixed carrier, i.e. a chip. A description of chips, that are used for the detection of mutations, may be found, for instance, in Warrington et al., “New Developments in High-Throughput Resequencing and Variation Detection Using High Density Microarrays.”, Human Mutation (2002): 19:402-409.A chip containing the corresponding allelic variants of at least one polymorphic gene region of the gene cited is brought into contact, for detection, with the nucleic acid specimens that are to be tested, after which the identity of the allelic variants is able to be ascertained via simple hybridization methods.

In one specific embodiment of the method according to the present invention, the PCR (polymerase chain reaction) method can be used, namely by using specific primers which flank the sequences that are of interest. In this method, first of all, nucleic acid sequences from a biological specimen of a patient/subject are isolated, these are contacted with the primers, in order to amplify a region that contains the corresponding SNP. The PCR product subsequently undergoes a second PCR reaction with a third primer flanking the SNP, which finally permits drawing conclusions on the allele present in the SNP position.

As an example, we refer to the standard work of Sambrock et al., “Molecular Cloning: A Laboratory Manual”, Cold Spring Harbor Laboratory Press, 1989, in which numerous methods for isolating, enriching and identifying sequences of interest are described.

The primers which, according to the exemplary embodiments and/or exemplary methods of the present invention, are used for detecting the SNP, are long enough in order specifically to hybridize to the target nucleotide sequence. In certain specific embodiments it may, in addition, be preferred if the primers have a marking, which may be, for instance, a radio isotope, a fluorescent compound, enzymes or the like. The primers used may be modified to the extent that their stability is increased, for instance, via the sugar group or the phosphate backbone.

The expression “hybridize under stringent conditions”, as it is used here, is meant to refer to hybridization conditions of primers on specific target sequences, under which no cross-hybridizations of polynucleotides to unrelated polynucleotides are possible; the stringent hybridization conditions may refer to an incubation at 43° C. in 50% formamide/10×SSC, followed by three rinsing steps in 1×SSC/0.2% SDS, 01,×SSC/0.2% SDS and 0.1×SSC.

A whole series of techniques and methods is known to one skilled in the art, by which the presence of certain SNP's in the candidate genes cited may be detected.

Therefore, the exemplary embodiments and/or exemplary methods of the present invention relate to a kit having a primer or a probe, using which the respective position of the SNP's cited herein are able to be detected or amplified, namely, in each case position 101 of a nucleic acid sequence, which is selected from the group made up of SEQ-ID-No. 1, SEQ-ID-No. 2, SEQ-ID-No. 3, SEQ-ID-No. 4, SEQ-ID-No. 5, SEQ-ID-No. 6, SEQ-ID-No. 7, SEQ-ID-No. 8, SEQ-ID-No. 9, SEQ-ID-No. 10, SEQ-ID-No. 11, SEQ-ID-No. 15, SEQ-ID No. 17, SEQ-ID No. 18, SEQ-ID No. 19, SEQ-ID-No. 20, SEQ-ID-No. 21, SEQ-ID-No. 22, SEQ-ID No. 23, SEQ-ID No. 24, SEQ-ID Nr. 25 or SEQ-ID No. 26, or Position 81 of a nucleic acid sequence, which is selected from the group made up of SEQ-ID-No. 12, SEQ-ID No. 13, SEQ-ID-No. 14, SEQ-ID-No. 16 or SEQ-ID No. 27.

In addition, the kit may also contain a detection arrangement and detection reagents, such as reagents for the hybridization of allele-specific oligonucleotides, for sequence-specific amplification, for a nuclease digestion, sequencing, primer extensions reactions, size determination, and others.

Moreover, the exemplary embodiments and/or exemplary methods of the present invention relate to the use of the SNP's cited herein, especially the SNP having Number rs2302685, for determining the predisposition of an organism for developing Crohn's Disease of the ileum having early onset.

In order to determine the predisposition of a person/a subject for developing Crohn's Disease, the polymorphism is detected, using the SNP's cited herein, in biological specimens of the person/the subject, and put into relationship with the control specimens, whereby the frequency of the allele may be put into proportion.

Therefore, using the exemplary embodiments and/or exemplary methods of the present invention, predictive methods are provided for the first time and possibilities based on the identification of the SNP's cited herein, which are associated with the probability that a subject will develop Crohn's Disease. The diagnostic information, gathered from this, is able to be used to determine how the respective subject may be treated specifically and in a timely manner, so as to prevent the disease, mitigate it or treat it, or in order to delay the onset of the disease.

According to the exemplary embodiments and/or exemplary methods of the present invention, the SNP's which were identified according to the present invention, may be used by themselves or in combination, to determine the predisposition of an organism for developing Crohn's Disease.

Further advantages of the new method will become apparent from the description and the attached figures. It is understood that the aforementioned features and the features still to be explained in the following text may be used not only in the respectively indicated combinations but also in other combinations or by themselves, without departing from the scope of the present invention.

Exemplary embodiments of the present invention are shown in the drawings.

### BRIEF DESCRIPTION OF THE DRAWINGS

FIG. 1a a map of the genomic region of the TCF4 gene having the localization of the SNP's drawn in, according to the present invention.

FIG. 1b a map of the genomic region of the TCF4 gene having localization drawn in of further SNP's (above); the allele frequency of the genetic variants is shown (below) in a bar graph.

FIG. 2 a map of a genomic region of the LPR5 gene having localization of the SNP's drawn in.

FIG. 3 a map of a genomic region of the LPR6 gene having localization of the SNP's drawn in.

FIG. 4 a summarizing, schematic overview of the effects of genetic changes in certain genes on the Paneth cell.

FIG. 5 the nucleic acid sequences of LRP6-SNP according to the present invention.

FIG. 6 the nucleic acid sequences of Tcf4-SNP according to the present invention.

FIG. 7 the nucleic acid sequences of LRP5-SNP according to the present invention.

### DETAILED DESCRIPTION

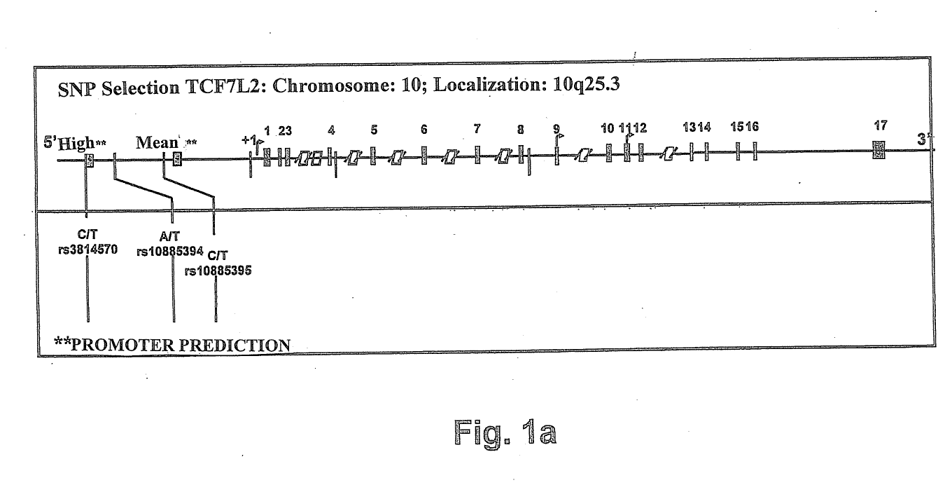

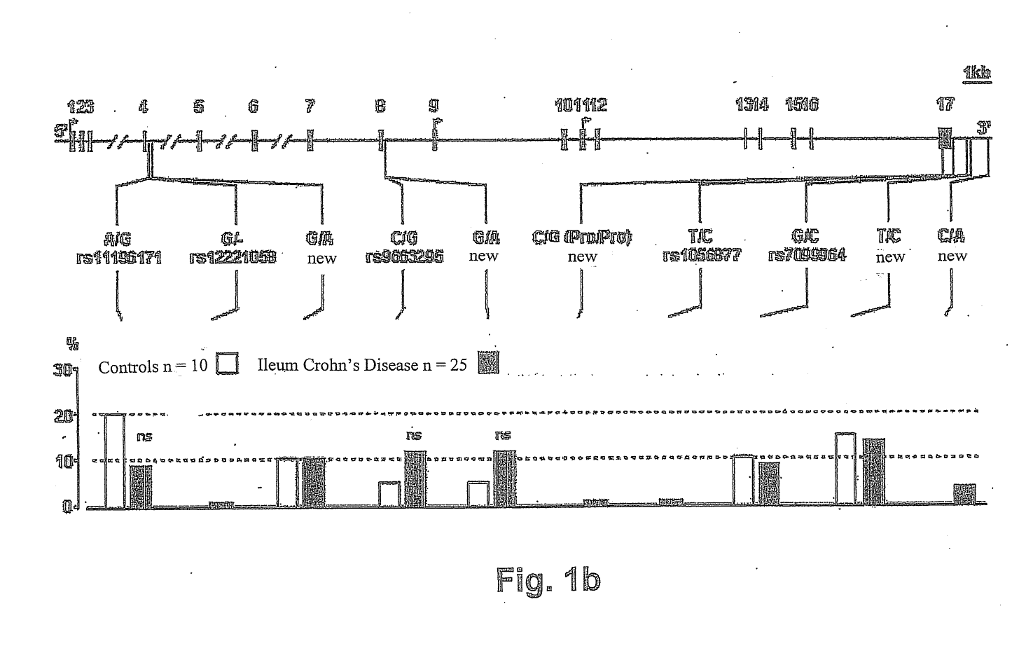

FIG. 1a shows the genetic map of TCF4, the positions of the identified SNP's being indicated. Number “1” designates SNP1 having rs Number rs3814570, “2” designates the SNP having rs Number rs10885394 and “3” designates the SNP having rs Number rs 10885395.

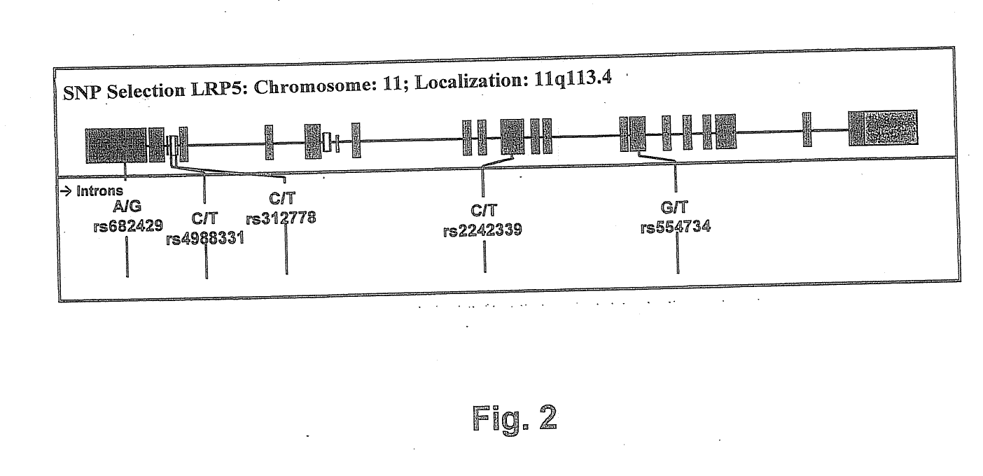

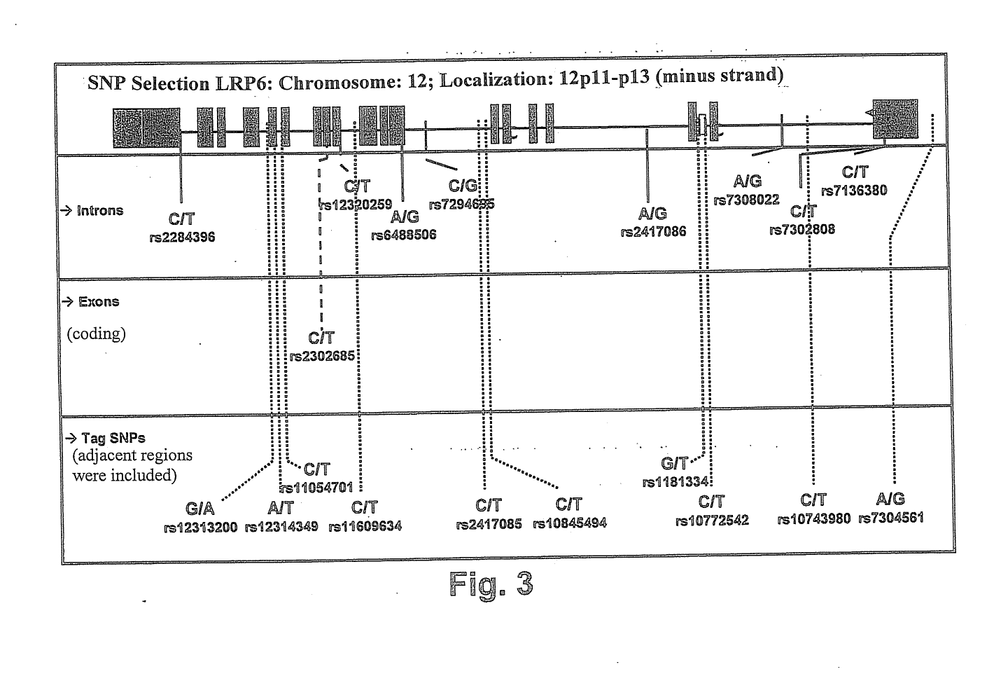

FIGS. 2 and 3 respectively show the genetic maps of LPR5 and LPR6, the respective positions of the SPN's in these genes being correspondingly given.

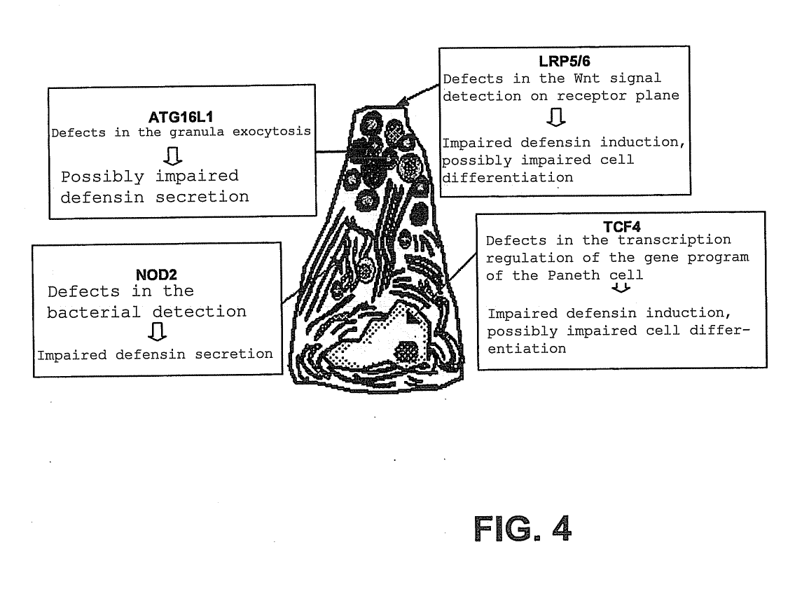

FIG. 4 shows a schematic representation of the effects of genetic changes on the Paneth cell. As is shown here, a schematic change in LPR5/6 leads to defects in the Wnt signal detection which, in turn, leads to a deterioration in the induction of the defensins, and possibly also to impaired cell differentiation. It is also shown that genetic mutations in TCF4 lead to defects in the transcription regulation of the gene program in Paneth cells which, in turn, leads to a clear deterioration in the induction of defensins, and possibly also to impaired cell differentiation.

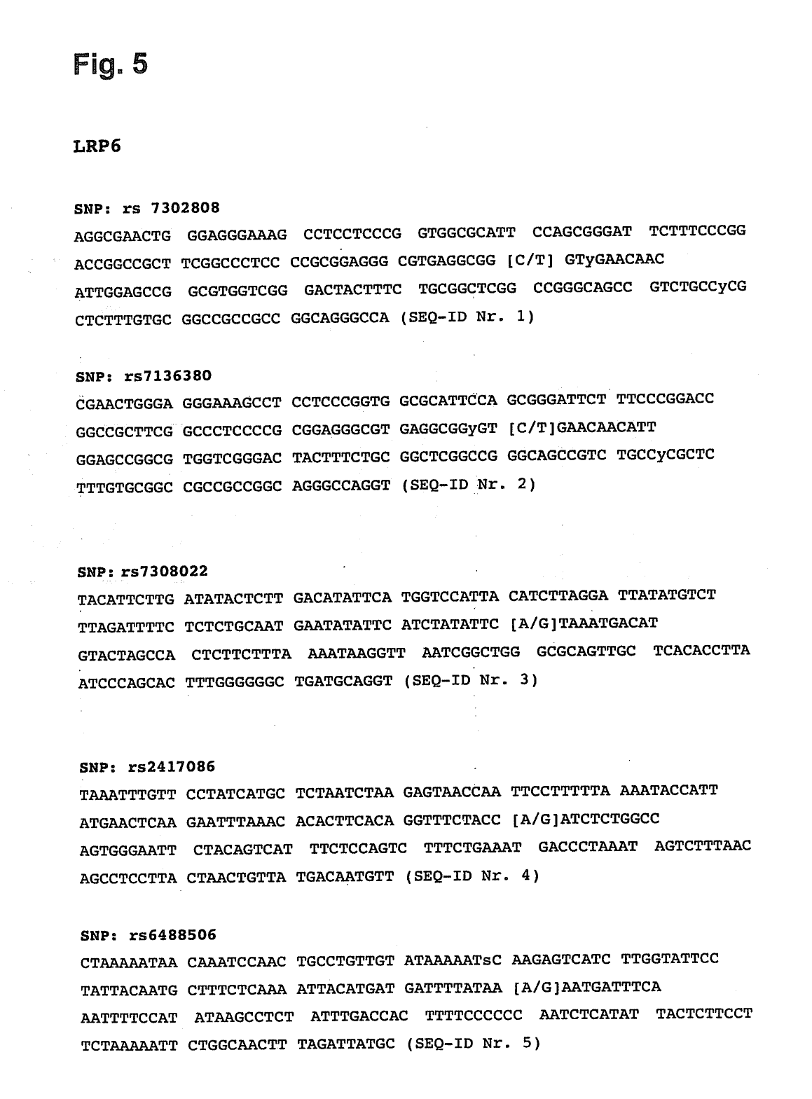

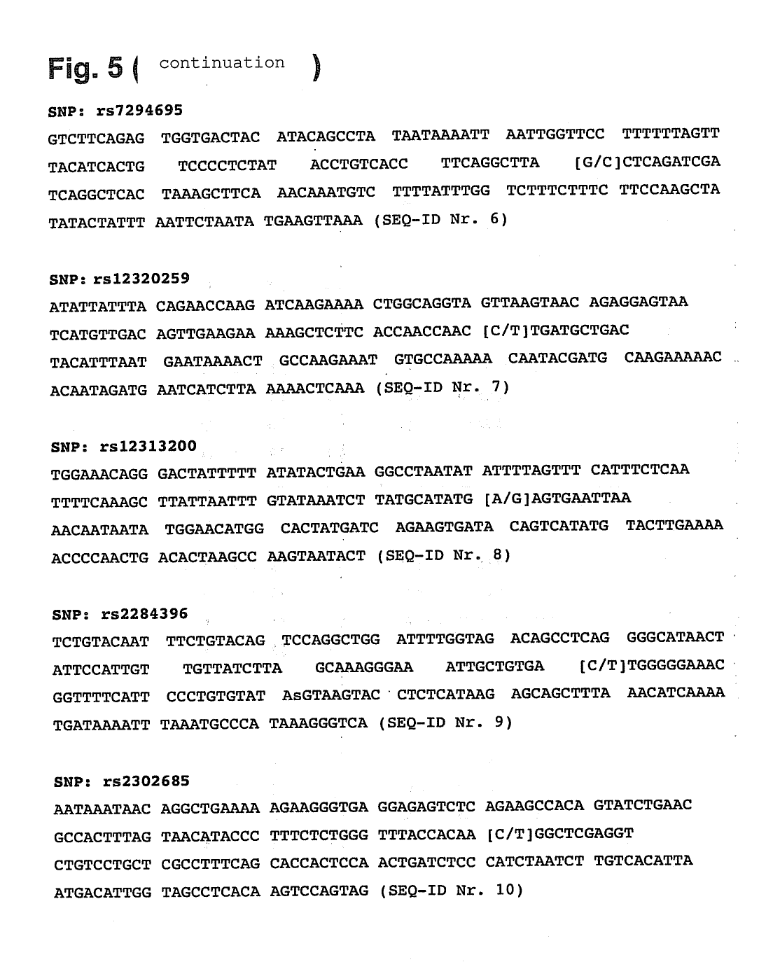

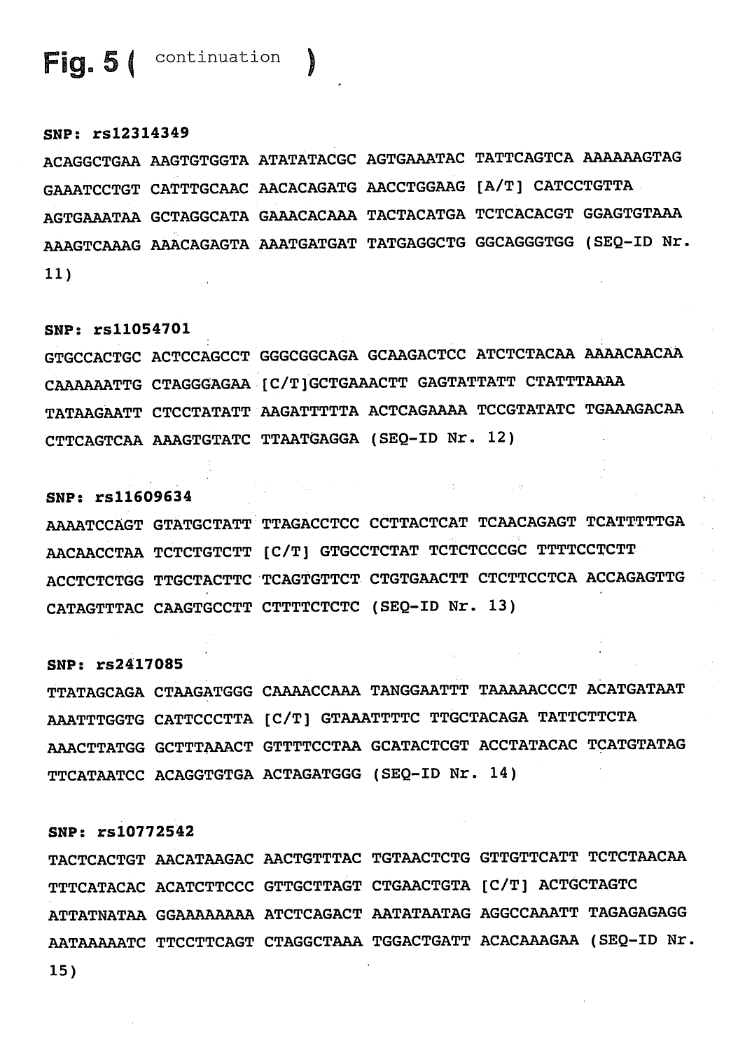

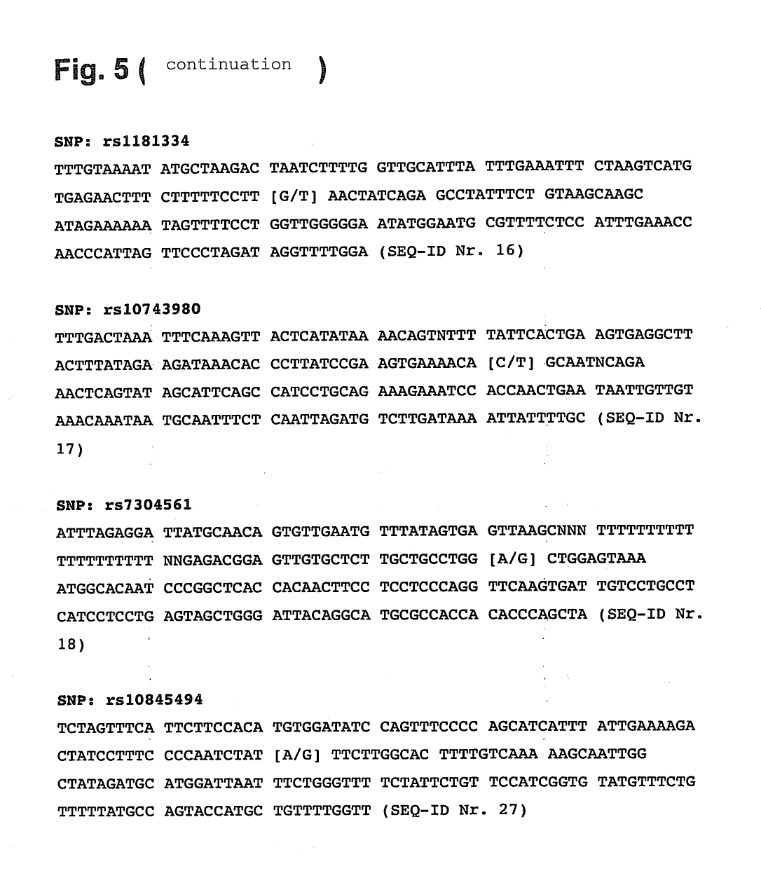

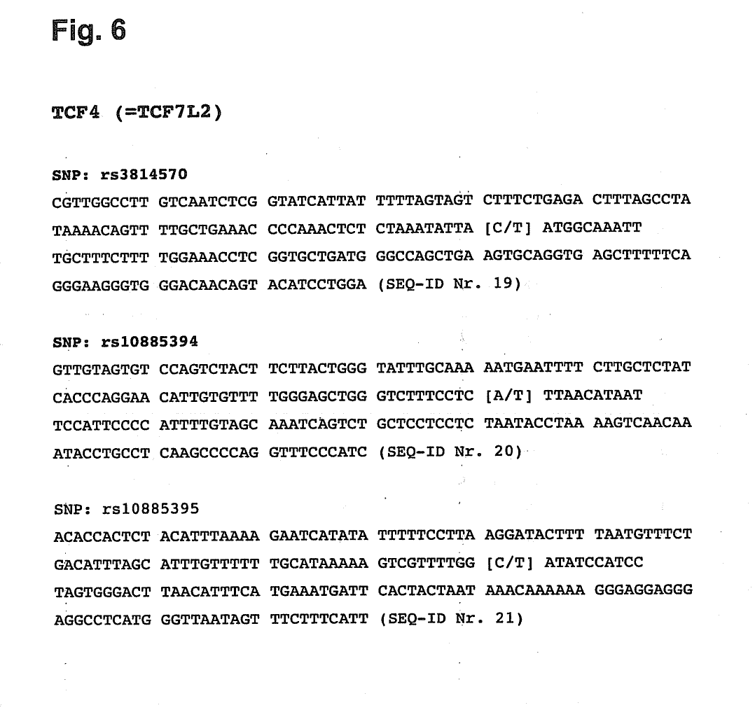

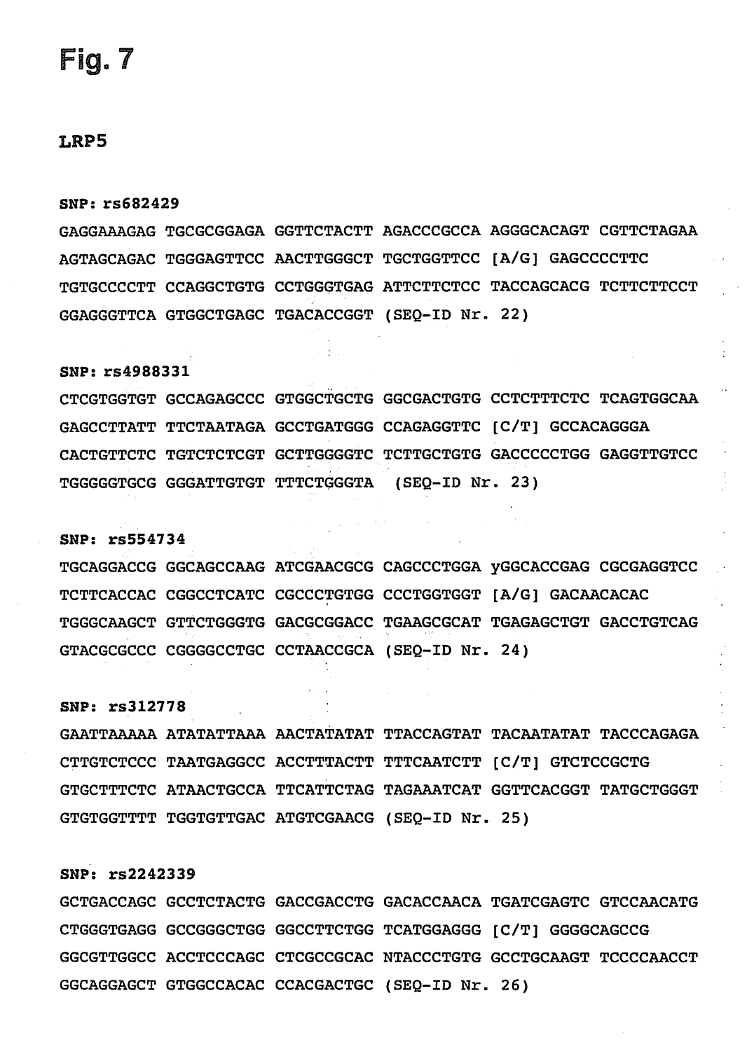

In FIGS. 5 through 7, the position of the SNP's, according to the present invention, is shown in the context of the nucleic acid sequences surrounding them, in FIG. 5 correspondingly the SNP in LRP6, in FIG. 6 the SNP in Tcf4 and in FIG. 7 the SNP in LRP5 being shown.

### Example 1

### Material and Methods

### a) Patients and Human Materials

For the genetic analysis, DNA specimens were taken from different patient cohorts: Patients of Caucasian descent having Crohn's Disease (N=259) or ulcerative colitis (N=149) from the University Clinic at Vienna; healthy blood donors of Caucasian descent not related to Group 1, from Stuttgart (N=833). For additional tests, DNA samples were collected from the following patient groups: Patients of Caucasian descent having Crohn's Disease (N=277) or ulcerative colitis (N=74) as well as healthy donors (N=242) of the University of Leuven, Belgium; a third cohort of patients of Caucasian descent having Crohn's Disease (N=473) or ulcerative colitis (N=562) as well as healthy donors (N=324) from Oxford. According to the classification of Montreal, three subgroups were defined for patients having Crohn's Disease: Patients having disease exclusively of the small intestine (L1), patients having disease exclusively of the large intestine (L2), patients having disease both of the small and the large intestine (L3) as well as patients having additional disease of the upper gastrointestinal tract. To put a value on the frequency distribution of certain SNP's, evaluations were made of a total of tests of 225 patients having Crohn's Disease exclusively in the large intestine (L2), 784 Crohn's Disease patients having at least partial involvement of the small intestine (L1+L3) as well as 785 patients having ulcerative colitis and altogether 1399 donors selected at random for Control. In order to exclude greater differences between the groups, with regard to the composition as to age and sex, in the case of the Crohn's Disease patients and the control patients, a further subdivision was undertaken according to these two criteria.

Furthermore, the intensity and aggressiveness of the course of the disease in the individual case was investigated. To do this, it was further checked with the patients whether, and at what frequency an inflammatory, structuring or penetrating intensity of the disease was appearing. The connection with Crohn's Disease-caused surgical interventions was also investigated.

The study was approved by the ethics committees of the Medical University Wien, Austria, the University Hospital Tubingen, Germany, the University at Leuven, Belgium and Oxford's Radcliffe Hospital Trust. After intensive clarification, all patients gave a written consent declaration for the analysis of their DNA within the scope of the present study.

### b) Sequencing of the Tcf-4 Promoter and the Tcf-4 Gene Region

In order to determine genetic variations in the Tcf-4 promoter, the 2.1 kb upstream region of healthy patients selected randomly (n=10) as well as those of patients having Crohn's Disease in the small intestine (n=10) were sequenced. In addition, the region of the Tcf-4 gene was sequenced in which, in the literature, functional insertions and deletions were observed in cancer of the intestine. Next, a sequence analysis of known Tcf-4 exons was carried out, inclusive of −100 by flanking introns, in order additionally to identify possible variants of this gene in these regions. The primers were selected while using ENSG00000148737 of the “Ensemble genome browser” data bank for the sequencing of the promoters and the exons. The sequencing was carried out according to standard methods.

### c) Genotyping

Leucocyte DNA was isolated from whole blood samples according to a standard method (QIAämp DNA Blood Mini Kit, Qiagen, Hilden, Germany). For the genotyping, the matrix assisted laser desorption/ionization time-of-flight (MALDITOF)-based mass spectrometric (MS) analysis of allele-specific primer extension products was used in a system by Bruker (Daltonik, Leipzig, German). In a subgroup of samples, the presence of SNP's, detected using MALDI-TOF MS, was checked by TaqMan®-Analyse und direct sequencing. The genotyping, based on MALDI-TOF MS, of DNA samples was carried out using MassARRAY® Compact Systems of Sequenom (San Diego, USA).

### d) Computer Analysis and Statistics

Using “Promoter 2.0: for Detecting PollI Promoter Sequences”, an in silico screening was carried out of a 10 kb Tcf-4 upstream region. The TESS (transcription element searching system) data bank-software makes it possible to investigate potential binding sites for certain transcription factors in the potentially interesting sequences. Using the Finetti-specialized software (http.//ing2.helmholtz-muenchen.de/cgi-bin/hw/hwal.pl), the polymorphisms were tested for the Hardy-Weinberg equilibrium, by using the log-likelihood ratio/chi square test in the three cohorts. For the genetic analysis (compare the IBD subgroups as opposed to the controls), this software was used in order to calculate nonlinear ratios, confidence intervals (C.I.), and to carry out “Pearson's goodness-of-fit chi-square tests”. The differences in the genotype frequencies were the subject of both t-tests and Armitage trend tests. Values under 0.05 were regarded as being significant. The linkage disequilibrium between the Tcf4 SNP's and the haplotype blocks was calculated and identified by using Haploview. In order to exclude a random association of SNP rs3814570, the significance of the p-values <0.05 was verified by using the correction according to Benjamini-Hochberg in the overall group.

### e) NOD2-Genotype Analysis

The genotyping of the usual NOD2 variants (SNP8, SNP12 and SNP13) was carried out by using the “TaqMan” technology (Applied Biosystems, Foster City, Calif., USA), as described above (9).

### Example 2

### Tcf-4

### a) SNP Selection and Haplotypes

To investigate the possible genetic linkage of Tcf-4 with ileum-CD, SNP's were screened with respect to the sequencing of 2.1 kb of the 5′ flanking region of Tcf-4, namely in a random group of 10 ileum CD patients and 10 healthy controls. In this assumed promoter region (see FIG. 1) eight SNP's were found, of which three (rs3814570, rs10885394, rs10885395) were in linkage disequilibrium (LD) in both the patient group and the control group. In the control group, two out of ten individuals were heterozygous with respect to these variants; in patients having ileum CD, six out of ten individuals were heterozygous. Based on these results, a well-investigated cohort of patients having CD as well as healthy controls from Vienna (Austria) were examined. Both in the control and in the CD group, an LD was found between the three SNP's, via which a new haplotype block was defined (see FIG. 2).

An in silico analysis of the promoter and the transcription factor binding sites of the sequenced region yielded a potential regulatory region near the localization of rs3814570. Based on (i) the observed reduced expression of the Tcr-4 mRNA, (ii) the greater frequency of the promoter variants and based on (iii) the presence of an assumed regulatory locus, the hypothesis was tested that rs3814570 demonstrates a greater frequency in patients having CD of the small intestine. In order to exclude greater variants in the gene region and possible LD of the identified promoter SNP's having other potential functional variants in the Tcf-4 gene, known coding exons were sequenced having approximately 100 kb overlapping intron borders in 10 randomly selected controls (6 identical to the promoter analysis) as well as in 25 patients having ileum CD (7 identical to the promoter analysis). Ten additional assumed SNP's were found, of which two were in LD, but none showed an LD having the promoter SNP's described. Based on the data published by the HapMap Project, a further search for haplotypes in Tcf-4 was carried out, and no haplotype block could be identified that included rs3814570 or additional SNP's in the gene region.

b) A Tcf-4 Promoter Variant is Associated with the Ileum CD Predisposition

The analysis of the frequency distribution of SNP rs3814570 was carried out in a total of 1399 controls (T-allele frequency=25.59%), as well as 785 UC patients (T-allele frequency=25.22%), 225 CD patients having L2 classification (T-allele frequency=24.67%) and 784 CD-patients having ileum involvement (L1+L3) (T-allele frequency=29.66%). By contrast to UC (OR 0.981, 95% Cl 0.851 to 1.131, n.s., which was similar to the controls), the CD patients combined showed a weak association for the variant (T-allele positivity: OR 1.182, 95% Cl 1.005 to 1.391, p=0.04358).

TABLE 3
Summary of the Origin of the Samples

| | Controls | UC | CD (L1) | CD (L2) | CD (L3) |
|---|---|---|---|---|---|
| Vienna | 833* | 149 | 54 | 55 | 150 |
| Leuven | 242 | 74 | 81 | 45 | 151 |
| Oxford | 324 | 562 | 94 | 125 | 254 |

*healthy blood donors from Stuttgart

In agreement with the initial hypothesis, the investigation of the different CD subgroups showed an association of the smaller variant (T) with ileum CD (OR 1,226, 95% Cl 1.068-1.407, p=0.00371), but not with large intestine CD (OR 0.952, 95% Cl 0.756 to 1.199, n.s.). The test with respect to the allele positivity by analysis of wild type homozygous individuals (CC) compared to all carriers of the small variant (CT+TT) showed a clearer effect during comparison of the healthy controls compared to ileum CD (OR 1.271, 95% Cl 1.066 to 1.515, p=0.007372).

Since there were differences in the allele frequencies between the different cohorts, it was checked whether these obvious frequency differences were statistically significant. In general, the Oxford cohort showed a low T-allele frequency in controls (23.30%) compared to the Leuven cohort (26.65%) as well as to the Vienna cohort (26.17%). The same applied to CD patients (T-allele frequency in Oxford: 27.38%, Leuven: 30.14% and Vienna 28.96%), however, this could be explained partially by the different percentage of large intestine CD patients in the groups. For CD having only ileum involvement, the frequency distributions in the cohorts were: rather more similar (T-allele frequency in Oxford: 28.30%, Leuven: 30.82% and Vienna 30.64%) and were not significantly different. Although we found a possible change in the frequency distributions between the Oxford control group and both the Leuven controls (allele frequency: OR 1.196, 95% Cl 0.912 to 1.569, p=0.19618) and the Vienna controls (allele frequency: OR 1.167, 95% Cl 0.943 to 1.443, p=0.15453), the differences were not significant. Therefore, an increased SNP frequency was shown in ileum CD patients in three independent European cohorts, and a uniquely significant association of the small variants could be observed for rs3814570 with ileum CD in the combined analysis of all samples.

c) The Association of rs3814570 with Ileum CD is Independent Of Sex, but Somewhat More Pronounced in Patients >40 Years Old

In order to make sure that there is no disorganization with respect to age and sex, all controls and CD patient groups were subgrouped according to these criteria. There were no agreeing differences with respect to allele frequency between men and women in both the controls and the patients; thus a sex-specific effect of the variants was able to be excluded. Interestingly enough, an increased association of the variant was found in the comparison of patients having ileum CD, but not in the case of exclusive large intestine CD, age group A3 (>40 years) having controls of the same age group in the overall analysis, nor in two separate cohorts (Leuven and Oxford). In the overall analysis, there was a statistical significance for homozygous carriers (homozygous carriers: OR 2.023, 95% Cl 1.010 to 4.052, p=0.04347).

### d) Rs3814570 Shows the Highest Frequency in Patients Having Stricturing Ileum Crohn's Disease

According to their occurrence, the patients were placed in groups 81 (inflammatory), B2 (stricturing) and B3 (piercing). In the overall analysis, the greatest frequency was found within the ileum CD subgroup having stricturing appearance (T-allele frequency: 31.25%). This was also obvious in two separate cohorts (T-allele frequency in Oxford: 29.81%, Leuven: 35.83%, but not in L2 CD patients. The association of the SNP with structuring ileum CD in comparison with healthy controls showed an high significance in the overall analysis (allele frequency: OR 1.322, 95% Cl 1.079 to 1.619, p=0.00686), and an additionally increased quantity of homozygous carriers was observed (homozygous carriers: OR 1.708, 95% Cl 1.107 to 2.634, p=0.01460). In order to identify a possible association with the aggressiveness of the disease, the patients were grouped into those that had at least one surgical intervention with respect to CD, and those that did not. No agreeing results were observed; although in two cohorts, there was a trend with respect to an higher frequency in the ileum CD group having surgical intervention (T-allele frequency in Oxford: 28.93% and Leuven: 31.58%) and a significantly stronger association with ileum CD in the group having surgical intervention in comparison to controls in a cohort (Oxford allele frequency: OR 1.340, 95% Cl 1.030 to 1.742, p=0.02885).

### e) Rs3814570 Confers the Risk of an Additional L4 Phenotype in Patients Having Ileum CD

In order specifically to address the question of an upper GIT involvement (L4), the patient groups were subdivided into two further subgroups according to this additional phenotype. In general, the quantity of patients having upper GIT involvement was rather low: Leuven patients having additional L4 phenotype: 12 patients L3; 4 patients L2; 6 patients L1; Oxford patients having additional L4 phenotype: 36 patients L3; 4 patients L2; 10 patients L1; Vienna patients having additional L4 phenotype: 40 patients L3; 10 patients L2; 11 patients L1. In the comparison of the allele frequency with the controls, we were able to find a slight rise in patients having ileum CD having an additional L4 phenotype (T-allele frequency: 32.17%). This, however, did not contribute to the L2 patients having upper GIT involvement. The stronger association of the rare variant was thus statistically significant in the overall analysis (allele frequency: OR 1.379, 95% Cl 1 to 1.033 to 1.842, p=0.02882).

### f) Rs3814570 is Independent of NOD2

On the assumption that 1007fsinsC-mutation (SNP13) in NOD2 is a known susceptibility factor for CD of the ileum, and that it is associated with reduced levels of HD5 and HD6, it was investigated whether the observed association of rs3814570 with ileum CD was independent of NOD2 in the Vienna and the Leuven cohorts. It had been reported before that the effects of reduced Tcf-4 on Paneth cells were defensins in ileum CD patients independent of the effects of SNP13 NOD2 variant, since these patients having this NOD2 mutation demonstrated (40) a clearly more pronounced reduction in HD-5 and HD-6 expression. The independence of these factors makes it obvious that the exclusion of patients, that carry NOD2 SNP13, should achieve similar allele frequencies of rs3814570 in the remaining ileum CD patients. In the comparison of all Leuven ileum CD patients (n=232) having a subgroup in which patients having SNP13 (n=191) were excluded, there were indeed no differences in the allele frequency (OR 0.991) or allele positivity (OR 0.984). The same applied to the Vienna ileum CD patients (ns=204): after. SNP13 exclusion (n=154) the allele frequency resulted in an OR of 1.057 and an allele positivity of OR 1.040. Thus the exclusion of patients having the NOD2 frameshift mutation SNP13 does not change the observed allele frequencies of rs3814570 in patients having ileum CD, which supports the different effects of this Tcf-4-SNP and NOD2 SNP13 in ileum CD.

Using the results attained, it could be shown that, using the new method, one is able to achieve an high probability of prognosis for Crohn's Disease. In addition, the results of the abovementioned statistical analyses show that, using the new method, it is possible, based on the presence or absence of various SNP's, particularly in the 5′-UTR of the TCF-4 gene, to predict a probability for the appearance of certain intensities of falling ill with Crohn's Disease.

The present results show that genes, that occupy important functions in the Wnt signaling pathway, demonstrate changes in connection with the disease. Since the signaling pathway is especially important for the proper development of the Paneth cell, factors involved with this are excellent target objects in the investigation of the malfunctioning of Paneth cells. The available data enable one uniquely to conclude that factors that codetermine the functioning of Paneth cells represent important candidates in the pathogenesis of Crohn's Disease. Thus, at present, genetic changes that are in connection with Crohn's Disease of the small intestine, have been identified with the proof for Tcf-4, the Wnt transcription factor which directly induces HD-5 and HD-6, as well as for low density lipoprotein receptor-related protein LRP5 and LRP6, important receptor molecules of the signaling pathways.

Consequently, the new method is a valuable tool for the prognosis of the appearance of chronic inflammatory bowel diseases as well as the individual diagnosis of the genetic effects on which such diseases are based.

### Example 3

### LRP6

### Material and Methods

As samples, the same material was used as in Example 1, and the same methods were used for their investigation as in Example 1.

### a) LRP6

The selection of LRP6 as the candidate gene was based on its specific and tissue-independent reduction in patients that have fallen ill. In order to cover variants, associated with the disease, in the intron region as well as the 3′ region and the 5′ region in the analysis, SNP's of the coding region and tag SNP's were included. The selection of tag SNP's was carried out using a “pairwise method”, by using the tag SNP selection function of the international “HapMap” Project Homepage (http://hapmap.ncbi.nlm.nih.gov/index.html.en). The aim of the international HapMap Project is to cartograph the haplotypes of the human genome. In order also to include promoter variants and other SNP's up or downstream of LRP6, we zoomed out outside the gene region (10% ca. 7.3 kb (kb=kilobases).

### b) LPR6 Genotyping

Leucocyte DNA was isolated using standard methods (such as described in Example 1; material and methods described in c).

### c) Computer Analysis and Statistics

The mRNA expression levels of different factors were analyzed using Graph-Pad Prism Software, Version 4.0 (see www.graphpad.com). Analyses that were non-parametric or parametric (in the case of a normal distribution) were carried out, namely using the U-Test of Wilcoxon, Mann and Whitney or the “Student t-Tests”, to compare the grouped data. P-values of less than 0.05 were regarded as statistically significant. For correlation tests, the “Spearmen-Rank” Analysis was carried out to investigate relationships between different gene products. Average values were shown in the figures with their corresponding standard errors. Using the Finetti-specialized software (http.//ing2.helmholtz-muenchen.de/cgi-bin/hw/hwal.pl), the polymorphisms were tested with respect to the Hardy-Weinberg equilibrium, by using the three cohorts of the log-likelihood ratio/chi-square test. For the genetic analysis (compare the IBD subgroups as opposed to the controls), this software was used in order to calculate nonlinear ratios, confidence intervals (C.I.), and to carry out “Pearson's goodness-of-fit chi-square tests”. The differences in the genotype frequencies were the subject of both t-tests and Armitage trend tests. Values under 0.05 were regarded as being significant. The linkage disequilibrium between the Tcf4 SNP's and the haplotype blocks was calculated and identified by using Haploview.

### d) The LRP6 Expression is Reduced in Ileum CD in a Tissue-Independent Manner

The mRNA levels of LRP5, LRP6, HD-5 and HD-6, interleukin-8 (IL-8) and of GapDH (glycerinaldehyde-3-phosphate-dehydrogenase) were tested in ileum and colon biopsy samples (data not shown). All the controls and patients had as genotype the NOD2 wild-type, so as to avoid distorted effects based on additionally reduced defensin levels in NOD2 SNP13 mutated individuals. In the GapDH expression no significant differences showed up between the groups, both in the ileum and the colon group. What was observed, however, was a strong and significant reduction of the LRP6-mRNA level in CD patients having involvement of the small intestine. In the ileum samples, the effect could be observed both in patients having an L1 phenotype (small intestine only), and in patients in which the colon was additionally involved (L3); however, the effect could not be observed in patients having exclusively colon involvement (L2). In the case of LRP5, about which it is known that it is generally expressed together with LRP6, this significant reduction in the mRNA expression was missing. Within the samples, the mRNA levels of LRP6 and HD5 showed an high degree of correlation. In the following, the LRP6 expression in Colon samples of healthy individuals, of patients having colitis indeterminata and having inflammatory bowel disease (IBD) were investigated. In the colon, no differences could be detected with respect to the inflammation in the LRP6 expression. This and the tissue-non-specific reduction of the factor in the L1 subphenotype speak against a secondary effect in the ileum of patients having small intestine CD.

e) A Rare Coding Variant of LRP6 is Associated with a Phenotype having Early Onset in Ileum CD.

Subsequently, frequency distributions and linkage disequilibria of the selected LRP6 SNP's were investigated in a well defined Oxford cohort which included almost 2000 DNA samples, and which were grouped into healthy controls and IBP patients. An association was found of the rare coding variants of rs2302685 with patients having ileum CD, which quite specifically had a phenotype having early onset. In agreement with these results, this specific association could be confirmed by subsequent analyses of two additional cohorts from Leuven and Vienna. Table 4 shows an overview of these results:

TABLE 4
Frequency Distribution and Genotypes

| | | Age at Diagnosis | | | | |
|---|---|---|---|---|---|---|
| | | 18 or | Up to and | Progress of Disease | | |
| Overview Ileum CD | | more | incl. 17 | Inflamm. | Strictured | Penetr. |
| Genotypes (n) | CC | 452 | 48 | 98 | 201 | 198 |
| | CT | 228 | 35 | 58 | 108 | 126 |
| | TT | 21 | 10 | 5 | 9 | 16 |
| Samples (n) | all | 701 | 93 | 161 | 318 | 340 |
| Genotypes | CC | 64.48% | 51.61% | 60.87% | 63.21% | 58.24% |
| (Frequency) | CT | 32.52% | 37.63% | 36.02% | 33.96% | 37.06% |
| | TT | 3.00% | 10.75% | 3.11% | 2.83% | 4.71% |
| Allele | C | 80.74% | 70.43% | 78.88% | 80.19% | 76.76% |
| f requency | | | | | | |
| | T | 19.26% | 29.57% | 21.12% | 19.81% | 23.24% |

A combination of all tested samples showed that an association in the ileum CD phenotype points to an early onset of the disease and a penetrating behavior to an influence of the variant on the progress of the disease and its severity, whereas the sex of the patients has no influence on the frequency distribution in the case of ileum CD.

It was further tested whether a dose dependence of the variant, as is shown in the illustration of the effects between homozygous and heterozygous, reinforces the role of the variant in ileum CD having early onset. It turned out that this is the case, since the subphenotyped group included an extremely large quantity of homozygous carriers, which goes along with a significantly increased risk for the corresponding homozygous genotype.

In order to investigate the role of LRP6 in the regulation of the alpha-defensin-mRNA expression in Paneth cells, their levels were investigated corresponding to the LRP6 rs2302685 genotype in the patients. For this purpose, the patients and the controls of the study were genotyped, and the samples were regrouped into LRP6 “mutant” LRP6 and wild-type.

Since it is known that an NOD2 frameshift mutation influences HD5 and HD6-mRNA levels, the effect of this mutation was also investigated as comparison. It showed (data not shown), that ileum CD patients having an heterozygous or homozygous mutated LRP6 genotype had the lowest HD5 level. The effect of the seldom coding LRP6 variant on the HD5-mRNA expression was comparable, though somewhat clearer than the effect of NOD2 SNP13 frameshift mutation, that had previously been brought into contact with reduced Paneth cell alpha-defensin levels. The expression level of LRP6 were reduced in ileum CD patients, and, to be sure, independent of the NOD2 or LRP6 genotype.

The results with respect to LRP6 show that the latter is a new and relevant Wnt signaling pathway factor in ileum CD. In the respective ileum of CD patients, LRP6 is impaired at the transcription level. The expression levels of LRP6 and HD5 were strongly correlated, and an LRP6 mutation, which is associated with an early onset of the disease, further reduced the alpha-defensin expression in Paneth cells in the case of ileum CD. The genetic connection and the tissue-nonspecific reduction in LRP6 point to a primary and extraordinary function of the coreceptor in the disease and confirm it.

1-20. (canceled)

21. A method for determining a predisposition of an organism for the development of Crohn's Disease, the method comprising:
determining, in a biological specimen of the organism, a presence of at least one single nucleotide polymorphism (SNP),
wherein the at least one SNP is determined in at least one gene, which codes for a protein associated with the Wnt signaling pathway in Paneth cells.

22. The method of claim 21, wherein the predisposition of an organism for Crohn's Disease of the small intestine is determined.

23. The method of claim 21, wherein the at least one gene is selected from the group including TCF4, LRP6, LRP5, GSK3A, GSK3B and TCF7.

24. The method of claim 23, wherein a nucleotide is determined at least one of the following positions relative to the start codon of the human TCF-4 gene, and wherein it is detected in the presence or the absence of at least one SNP: −2006, −1728, −1248.

25. The method of claim 23, wherein a nucleotide is determined at one of the positions relative to the start codon of the human LRP5 gene, and wherein it is detected in the presence or the absence of at least one SNP: −864, 5150, 112335, 28149, 98942.

26. The method of claim 23, wherein a nucleotide is determined at least one of the positions relative to the start codon of the human LRP6 gene, and wherein it is detected in the presence or the absence of at least one SNP: −563, −566, 13797, 69556, 101286, 96051, 119500, 135079, 144734, 117771, 134754, 131848, 130513, 109982, 88294, 25774, 52081, 70770, 6874, −2736.

27. The method of claim 21, wherein the determination of the presence or the absence of the at least one SNP takes place by the primer extension of a PCR product and MALDI-TOF analysis.

28. The method of claim 21, wherein the determination of the presence or the absence of the at least one SNP takes place by the sequencing of a PCR product.

29. The method of claim 21, wherein the determination of the presence or the absence of the at least one SNP takes place by the hybridization of a PCR product to an allele-specific polynucleotide probe under stringent conditions.

30. An oligonucleotide probe, comprising:
between 5 and 50 nucleotides; and
a detecting arrangement to detect an SNP in one of the genes TCF4, LRP5, LRP6, GSK3A, GSK3B, and TCF7,
wherein the SNP is selected from rs3814570, rs10885394, rs10885395, rs682429, rs4988331, rs554734, rs312778, rs7302808, rs7136380, rs7308022, rs2417086, rs6488506, rs7294695, rs12320259, rs1231320, rs2284396, rs2302685, 12314349, rs12313200, rs11054701, rs11609634, rs2417085, rs10845494, rs1181334, rs10772542, rs10743980, rs7304561, for determining a predisposition of an organism for the development of Crohn's Disease, by performing the following:
determining, in a biological specimen of the organism, a presence of at least one single nucleotide polymorphism (SNP), wherein the at least one SNP is determined in at least one gene, which codes for a protein associated with the Wnt signaling pathway in Paneth cells.

31. The oligonucleotide probe of claim 30, wherein, when using it, the position 101 of a nucleic acid sequence is able to be detected, which is selected from the group made up of SEQ-ID-No. 1, SEQ-ID-No. 2, SEQ-ID-No. 3, SEQ-ID-No. 4, SEQ-ID-No. 5, SEQ-ID-No. 6, SEQ-ID-No. 7, SEQ-ID-No. 8, SEQ-ID-No. 9, SEQ-ID-No. 10, SEQ-ID-No. 11, SEQ-ID-No. 15, SEQ-ID No. 17, SEQ-ID-No. 18, SEQ-ID No. 19, SEQ-ID-No. 20, SEQ-ID-No. 21, SEQ-ID-No. 22, SEQ-ID No. 23, SEQ-ID No. 24, SEQ-ID No. 25 or SEQ-ID No. 26.

32. The oligonucleotide probe of claim 30, wherein, when using it, the position 81 of a nucleic acid sequence is able to be detected, which is selected from the group made up of SEQ-ID-No. 12, SEQ-ID-No. 13, SEQ-ID-No. 14, SEQ-ID-Nr. 16 or SEQ-ID No. 27.

33. A diagnostic kit, comprising:
at least one primer for determining a predisposition of an organism for the development of Crohn's Disease, by performing the following:
determining, in a biological specimen of the organism, a presence of at least one single nucleotide polymorphism (SNP),
wherein the at least one SNP is determined in at least one gene, which codes for a protein associated with the Wnt signaling pathway in Paneth cells;
wherein the determination of the presence or the absence of the at least one SNP takes place by the primer extension of a PCR product and MALDI-TOF analysis.

34. A use of an SNP in at least one gene, which codes for a protein associated with the Wnt signaling pathway in Paneth cells, for determining a predisposition of an organism for chronic inflammatory bowel diseases.

35. The use of claim 34, wherein the inflammatory bowel disease is Crohn's Disease.

36. The use of claim 34, wherein the gene is selected from the group including TCF4, LRP6, LRP5, GSK3A, GSK3B and TCF7.

37. The use of claim 34, wherein an SNP is used, which is selected from the group including rs3814570, rs10885394, rs10885395, rs682429, rs4988331, rs554734, rs312778, rs7302808, rs7136380, rs7308022, rs2417086, rs6488506, rs7294695, rs12320259, rs1231320, rs2284396, rs2302685, 12314349, rs12313200, rs11054701, rs11609634, rs2417085, rs10845494, rs1181334, rs10772542, rs10743980, rs7304561.

38. The use of claim 34, wherein an SNP having the number rs2302685 is used in order to determine the predisposition of a patient for the development of Crohn's Disease of the ileum having early onset.

39. The method of claim 21, wherein at least one SNP is detected, which is selected from the group including rs3814570, rs10885394, rs10885395, rs682429, rs4988331, rs554734, rs312778, rs7302808, rs7136380, rs7308022, rs2417086, rs6488506, rs7294695, rs12320259, rs1231320, rs2284396, rs2302685, 12314349, rs12313200, rs11054701, rs11609634, rs2417085, rs10845494, rs1181334, rs10772542, rs10743980, rs7304561.

40. A method for determining a predisposition of an organism for the development of Crohn's Disease, for the differential diagnosis of inflammatory bowel diseases, for distinguishing Crohn's Disease and the other respective inflammatory or infectious intestinal diseases, the method comprising:
determining, in a biological specimen of the organism, a presence of at least one single nucleotide polymorphism (SNP),
wherein the at least one SNP is determined in at least one gene, which codes for a protein associated with the Wnt signaling pathway in Paneth cells.

41. A diagnostic kit, comprising:
an oligonucleotide probe, including:
between 5 and 50 nucleotides; and
a detecting arrangement to detect an SNP in one of the genes TCF4, LRP5, LRP6, GSK3A, GSK3B, and TCF7,
wherein the SNP is selected from rs3814570, rs10885394, rs10885395, rs682429, rs4988331, rs554734, rs312778, rs7302808, rs7136380, rs7308022, rs2417086, rs6488506, rs7294695, rs12320259, rs1231320, rs2284396, rs2302685, 12314349, rs12313200, rs11054701, rs11609634, rs2417085, rs10845494, rs1181334, rs10772542, rs10743980, rs7304561, for determining a predisposition of an organism for the development of Crohn's Disease, by performing the following:
determining, in a biological specimen of the organism, a presence of at least one single nucleotide polymorphism (SNP),
wherein the at least one SNP is determined in at least one gene, which codes for a protein associated with the Wnt signaling pathway in Paneth cells.


SEQUENCE LISTING (27 sequences)

SEQ ID NO: 1 - DNA, 201 bp, Homo sapiens
  misc_feature (101)..(101): y is t or c
aggcgaactgggagggaaagcctcctcccggtggcgcattccagcgggattctttcccggaccggccgcttcggccctccccgcggagggcgtgaggcggygtygaacaacattggagccggcgtggtcgggactactttctgcggctcggccgggcagccgtctgccycgctctttgtgcggccgccgccggcagggcca

SEQ ID NO: 2 - DNA, 201 bp, Homo sapiens
  misc_feature (101)..(101): y is t or c
cgaactgggagggaaagcctcctcccggtggcgcattccagcgggattctttcccggaccggccgcttcggccctccccgcggagggcgtgaggcggygtygaacaacattggagccggcgtggtcgggactactttctgcggctcggccgggcagccgtctgccycgctctttgtgcggccgccgccggcagggccaggt

SEQ ID NO: 3 - DNA, 201 bp, Homo Sapiens
  misc_feature (101)..(101): r is a or g
tacattcttgatatactcttgacatattcatggtccattacatcttaggattatatgtctttagattttctctctgcaatgaatatattcatctatattcrtaaatgacatgtactagccactcttctttaaaataaggttaatcggctgggcgcagttgctcacaccttaatcccagcactttggggggctgatgcaggt

SEQ ID NO: 4 - DNA, 200 bp, Homo Sapiens
  misc_feature (101)..(101): r is g or a
taaatttgttcctatcatgctctaatctaagagtaaccaattcctttttaaaataccattatgaactcaagaatttaaacacacttcacaggtttctaccratctctggccagtgggaattctacagtcatttctccagtctttctgaaatgaccctaaatagtctttaacagcctccttactaactgttatgacaatgt

SEQ ID NO: 5 - DNA, 201 bp, Homo sapiens
  misc_feature (101)..(101): r is a or g
ctaaaaataacaaatccaactgcctgttgtataaaaatscaagagtcatcttggtattcctattacaatgctttctcaaaattacatgatgattttataaraatgatttcaaattttccatataagcctctatttgaccacttttccccccaatctcatattactcttccttctaaaaattctggcaactttagattatgc

SEQ ID NO: 6 - DNA, 201 bp, Homo sapiens
  misc_feature (101)..(101): s or g or c
gtcttcagagtggtgactacatacagcctataataaaattaattggttccttttttagtttacatcactgtcccctctatacctgtcaccttcaggcttasctcagatcgatcaggctcactaaagcttcaaacaaatgtcttttatttggtctttctttcttccaagctatatactatttaattctaatatgaagttaaa

SEQ ID NO: 7 - DNA, 201 bp, Homo sapiens
  misc_feature (101)..(101): y is c or t
atattatttacagaaccaagatcaagaaaactggcaggtagttaagtaacagaggagtaatcatgttgacagttgaagaaaaagctcttcaccaaccaacytgatgctgactacatttaatgaataaaactgccaagaaatgtgccaaaaacaatacgatgcaagaaaaacacaatagatgaatcatcttaaaaactcaaa

SEQ ID NO: 8 - DNA, 201 bp, Homo sapiens
  misc_feature (101)..(101): r is a or g
tggaaacagggactatttttatatactgaaggcctaatatattttagtttcatttctcaattttcaaagcttattaatttgtataaatcttatgcatatgragtgaattaaaacaataatatggaacatggcactatgatcagaagtgatacagtcatatgtacttgaaaaaccccaactgacactaagccaagtaatact

SEQ ID NO: 9 - DNA, 201 bp, Homo sapiens
  misc_feature (101)..(101): y is c or t
cctgtacaatttctgtacagtccaggctggattttggtagacagcctcaggggcataactattccattgttgttatcttagcaaagggaaattgctgtgaytgggggaaacggttttcattccctgtgtatasgtaagtacctctcataagagcagctttaaacatcaaaatgataaaatttaaatgcccataaagggtca

SEQ ID NO: 10 - DNA, 201 bp, Homo sapiens
  misc_feature (101)..(101): y is c or t
aataaataacaggctgaaaaagaagggtgaggagagtctcagaagccacagtatctgaacgccactttagtaacataccctttctctgggtttaccacaayggctcgaggtctgtcctgctcgcctttcagcaccactccaactgatctcccatctaatcttgtcacattaatgacattggtagcctcacaagtccagtag

SEQ ID NO: 11 - DNA, 221 bp, Homo sapiens
  misc_feature (101)..(101): w is a or t
acaggctgaaaagtgtggtaatatatacgcagtgaaatactattcagtcaaaaaaagtaggaaatcctgtcatttgcaacaacacagatgaacctggaagwcatcctgttaagtgaaataagctaggcatagaaacacaaatactacatgatctcacacgtggagtgtaaaaaagtcaaagaaacagagtaaaatgatgattatgaggctgggcagggtgg

SEQ ID NO: 12 - DNA, 201 bp, Homo sapiens
  misc_feature (81)..(81): y is c or t
gtgccactgcactccagcctgggcggcagagcaagactccatctctacaaaaaacaacaacaaaaaattgctagggagaaygctgaaacttgagtattattctatttaaaatataagaattctcctatattaagatttttaactcagaaaatccgtatatctgaaagacaacttcagtcaaaaagtgtatcttaatgagga

SEQ ID NO: 13 - DNA, 201 bp, Homo sapiens
  misc_feature (81)..(81): y is c or t
aaaatccagtgtatgctattttagacctccccttactcattcaacagagttcatttttgaaacaacctaatctctgtcttygtgcctctattctctcccgcttttcctcttacctctctggttgctacttctcagtgttctctgtgaacttctcttcctcaaccagagttgcatagtttaccaagtgccttcttttctctc

SEQ ID NO: 14 - DNA, 201 bp, Homo sapiens
  misc_feature (33)..(33): n is a, c, g or t
ttatagcagactaagatgggcaaaaccaaatanggaattttaaaaaccctacatgataataaatttggtgcattcccttaygtaaattttcttgctacagatattcttctaaaacttatgggctttaaactgttttcctaagcatactcgtacctatacactcatgtatagttcataatccacaggtgtgaactagatggg

SEQ ID NO: 15 - DNA, 221 bp, Homo sapiens
  misc_feature (101)..(101): y is c or t
tactcactgtaacataagacaactgtttactgtaactctggttgttcatttctctaacaatttcatacacacatcttcccgttgcttagtctgaactgtayactgctagtcattatnataaggaaaaaaaaatctcagactaatataatagaggccaaatttagagagaggaataaaaatcttccttcagtctaggctaaatggactgattacacaaagaa

SEQ ID NO: 16 - DNA, 201 bp, Homo sapiens
  misc_feature (81)..(81): k is g or t
tttgtaaaatatgctaagactaatcttttggttgcatttatttgaaatttctaagtcatgtgagaactttctttttccttkaactatcagagcctatttctgtaagcaagcatagaaaaaatagttttcctggttgggggaatatggaatgcgttttctccatttgaaaccaacccattagttccctagataggttttgga

SEQ ID NO: 17 - DNA, 221 bp, Homo sapiens
  misc_feature (37)..(37): n is a, c, g, or t
tttgactaaatttcaaagttactcatataaaacagtnttttattcactgaagtgaggcttactttatagaagataaacacccttatccgaagtgaaaacaygcaatncagaaactcagtatagcattcagccatcctgcagaaagaaatccaccaactgaataattgttgtaaacaaataatgcaatttctcaattagatgtcttgataaaattattttgc

SEQ ID NO: 18 - DNA, 221 bp, Homo sapiens
  misc_feature (48)..(50): n is a, c, g, or t
atttagaggattatgcaacagtgttgaatgtttatagtgagttaagcnnnttttttttttttttttttttnngagacggagttgtgctcttgctgcctggrctggagtaaaatggcacaatcccggctcaccacaacttcctcctcccaggttcaagtgattgtcctgcctcatcctcctgagtagctgggattacaggcatgcgccaccacacccagcta

SEQ ID NO: 19 - DNA, 201 bp, Homo sapiens
  misc_feature (101)..(101): y is c or t
cgttggccttgtcaatctcggtatcattatttttagtagtctttctgagactttagcctataaaacagttttgctgaaaccccaaactctctaaatattayatggcaaatttgctttcttttggaaacctcggtgctgatgggccagctgaagtgcaggtgagctttttcagggaagggtgggacaacagtacatcctgga

SEQ ID NO: 20 - DNA, 201 bp, Homo sapiens
  misc_feature (101)..(101): w is a or t
gttgtagtgtccagtctacttcttactgggtatttgcaaaaatgaattttcttgctctatcacccaggaacattgtgttttgggagctgggtctttcctcwttaacataattccattccccattttgtagcaaatcagtctgctcctcctctaatacctaaaagtcaacaaatacctgcctcaagccccaggtttcccatc

SEQ ID NO: 21 - DNA, 201 bp, Homo sapiens
  misc_feature (101)..(101): y is c or t
acaccactctacatttaaaagaatcatatatttttccttaaggatacttttaatgtttctgacatttagcatttgttttttgcataaaaagtcgttttggyatatccatcctagtgggacttaacatttcatgaaatgattcactactaataaacaaaaaagggaggagggaggcctcatgggttaatagtttctttcatt

SEQ ID NO: 22 - DNA, 201 bp, Homo sapiens
  misc_feature (101)..(101): r is a or g
gaggaaagagtgcgcggagaggttctacttagacccgccaagggcacagtcgttctagaaagtagcagactgggagttccaacttgggcttgctggttccrgagccccttctgtgccccttccaggctgtgcctgggtgagattcttctcctaccagcacgtcttcttcctggagggttcagtggctgagctgacaccggt

SEQ ID NO: 23 - DNA, 201 bp, Homo sapiens
  misc_feature (101)..(101): y is c or t
ctcgtggtgtgccagagcccgtggctgctgggcgactgtgcctctttctctcagtggcaagagccttattttctaatagagcctgatgggccagaggttcygccacagggacactgttctctgtctctcgtgcttggggtctcttgctgtggaccccctgggaggttgtcctgggggtgcggggattgtgttttctgggta

SEQ ID NO: 24 - DNA, 201 bp, Homo sapiens
  misc_feature (101)..(101): r is a or g
tgcaggaccgggcagccaagatcgaacgcgcagccctggayggcaccgagcgcgaggtcctcttcaccaccggcctcatccgccctgtggccctggtggtrgacaacacactgggcaagctgttctgggtggacgcggacctgaagcgcattgagagctgtgacctgtcaggtacgcgccccggggcctgccctaaccgca

SEQ ID NO: 25 - DNA, 201 bp, Homo sapiens
  misc_feature (101)..(101): y is c or t
gaattaaaaaatatattaaaaactatatatttaccagtattacaatatattacccagagacttgtctccctaatgaggccacctttactttttcaatcttygtctccgctggtgctttctcataactgccattcattctagtagaaatcatggttcacggttatgctgggtgtgtggtttttggtgttgacatgtcgaacg

SEQ ID NO: 26 - DNA, 201 bp, Homo sapiens
  misc_feature (101)..(101): y is c or t
gctgaccagcgcctctactggaccgacctggacaccaacatgatcgagtcgtccaacatgctgggtgagggccgggctggggccttctggtcatggagggyggggcagccgggcgttggccacctcccagcctcgccgcacntaccctgtggcctgcaagttccccaacctggcaggagctgtggccacacccacgactgc

SEQ ID NO: 27 - DNA, 201 bp, Homo sapiens
  misc_feature (81)..(81): r is a or g
tctagtttcattcttccacatgtggatatccagtttccccagcatcatttattgaaaagactatcctttccccaatctatrttcttggcacttttgtcaaaaagcaattggctatagatgcatggattaatttctgggttttctattctgttccatcggtgtatgtttctgtttttatgccagtaccatgctgttttggtt